SEQUENCE LISTING (57 sequences)

SEQ ID NO: 1 - DNA, 12 bp, Clostridium sp.
  misc_feature 5, 6, 7, 8: n = A,T,C or G
gaacnnnngttc

SEQ ID NO: 2 - DNA, 14 bp, Artificial Sequence
  misc_feature 7: n = A,T,C or G
cgaacrnrygttyc

SEQ ID NO: 3 - DNA, 16 bp, Artificial Sequence
  misc_feature 4, 5, 6, 7, 8, 9, 10, 11, 12, 13: n = A,T,C or G
ctgnnnnnnnnnncag

SEQ ID NO: 4 - DNA, 14 bp, Bacillus subtilis
cgaatatgcgttcg

SEQ ID NO: 5 - DNA, 14 bp, Bacillus subtilis
cgaactttagttcg

SEQ ID NO: 6 - DNA, 14 bp, Bacillus subtilis
agaactcatgttcg

SEQ ID NO: 7 - DNA, 14 bp, Bacillus subtilis
cgaacgtatgtttg

SEQ ID NO: 8 - DNA, 14 bp, Bacillus subtilis
agaacaagtgttcg

SEQ ID NO: 9 - DNA, 14 bp, Bacillus subtilis
cgaacctatgtttg

SEQ ID NO: 10 - DNA, 14 bp, Bacillus subtilis
cgaacaaacgtttc

SEQ ID NO: 11 - DNA, 14 bp, Bacillus subtilis
ggaatgtttgttcg

SEQ ID NO: 12 - DNA, 14 bp, Bacteroides fragilis
cgaattaaactttg

SEQ ID NO: 13 - DNA, 14 bp, Bacteroides fragilis
cgaacggatcatcg

SEQ ID NO: 14 - DNA, 14 bp, Clostridium perfringens
agaacttatgttcg

SEQ ID NO: 15 - DNA, 14 bp, Corynebacterium glutamicum
cgtaggaattttcg

SEQ ID NO: 16 - DNA, 14 bp, Corynebacterium pseudotuberculosis
agaatggtcgttag

SEQ ID NO: 17 - DNA, 14 bp, Deinococcus radiodurans
cgatcctgcgtaag

SEQ ID NO: 18 - DNA, 14 bp, Mycobacterium leprae
cgaacagatgttcg

SEQ ID NO: 19 - DNA, 14 bp, Mycobacterium smegmatis
cgaacaggtgttcg

SEQ ID NO: 20 - DNA, 16 bp, Mycobacterium tuberculosis
tcgaacaggtgttcga

SEQ ID NO: 21 - DNA, 16 bp, Mycobacterium tuberculosis
tcgaacacatgtttga

SEQ ID NO: 22 - DNA, 14 bp, Staphylococcus aureus
cgaacaaatattcg

SEQ ID NO: 23 - DNA, 14 bp, Streptococcus mutans
cgaacatgcccttg

SEQ ID NO: 24 - DNA, 14 bp, Streptomyces lividans
cgaacatccattct

SEQ ID NO: 25 - DNA, 14 bp, Thermotoga maritima
cgaatgtcagtttg

SEQ ID NO: 26 - DNA, 20 bp, Escherichia coli
tactgtatgagcatacagta

SEQ ID NO: 27 - DNA, 20 bp, Escherichia coli
tactgtatattcattcaggt

SEQ ID NO: 28 - DNA, 20 bp, Escherichia coli
aactgtttttttatccagta

SEQ ID NO: 29 - DNA, 20 bp, Escherichia coli
tactgtacatccatacagta

SEQ ID NO: 30 - DNA, 20 bp, Escherichia coli
atctgtatatatacccagct

SEQ ID NO: 31 - DNA, 20 bp, Escherichia coli
tactgtataaataaacagtt

SEQ ID NO: 32 - DNA, 20 bp, Escherichia coli
tactgtgtatatatacagta

SEQ ID NO: 33 - DNA, 20 bp, Escherichia coli
tgctgtatatactcacagca

SEQ ID NO: 34 - DNA, 20 bp, Escherichia coli
aactgtatatacacccaggg

SEQ ID NO: 35 - DNA, 20 bp, Escherichia coli
tgctgtatataaaaccagtg

SEQ ID NO: 36 - DNA, 20 bp, Escherichia coli
cagtggttatatgtacagta

SEQ ID NO: 37 - DNA, 20 bp, Escherichia coli
tactgtatatgtatccatat

SEQ ID NO: 38 - DNA, 20 bp, Escherichia coli
tactgtatataaacacatgt

SEQ ID NO: 39 - DNA, 20 bp, Escherichia coli
acatgtgaatatatacagtt

SEQ ID NO: 40 - DNA, 20 bp, Escherichia coli
atctgtacataaaaccagtg

SEQ ID NO: 41 - DNA, 20 bp, Escherichia coli
tactgtatataaaaacagta

SEQ ID NO: 42 - DNA, 20 bp, Escherichia coli
tactgtatataaaaccagtt

SEQ ID NO: 43 - DNA, 20 bp, Escherichia coli
tactgtacacaataacagta

SEQ ID NO: 44 - DNA, 38 bp, Artificial Sequence
  Synthetic Primer
tatattgacaaatgaacaaatgttcatataattatatg

SEQ ID NO: 45 - DNA, 38 bp, Artificial Sequence
  Synthetic Primer
catataattatatgaacatttgttcatttgtcaatata

SEQ ID NO: 46 - DNA, 28 bp, Artificial Sequence
  Synthetic Primer
taattatatgtatagagagaaaggttgg

SEQ ID NO: 47 - DNA, 28 bp, Artificial Sequence
  Synthetic Primer
ccaacctttctctctatacatataatta

SEQ ID NO: 48 - DNA, 38 bp, Artificial Sequence
  Synthetic Primer
tttaagggactttgaacatatgttcttgacaaattaat

SEQ ID NO: 49 - DNA, 38 bp, Artificial Sequence
  Synthetic Primer
attaatttgtcaagaacatatgttcaaagtcccttaaa

SEQ ID NO: 50 - DNA, 18 bp, Artificial Sequence
  Synthetic Primer
gtaagatcaagtagtcaa

SEQ ID NO: 51 - DNA, 18 bp, Artificial Sequence
  Synthetic
cagagcaatgactccaaa

SEQ ID NO: 52 - DNA, 22 bp, Artificial Sequence
  Synthetic
ggagcaaacaggattagatacc

SEQ ID NO: 53 - DNA, 21 bp, Artificial Sequence
  Synthetic primer
tgccaactctatggtgtgacg

SEQ ID NO: 54 - DNA, 99 bp, Escherichia coli
ttaagggacttttattatattatattgacaaattaataaattactatataattatatgtatagaacaaatgttcgagagaaaggttggtgaacccttaa

SEQ ID NO: 55 - DNA, 100 bp, Clostridium acetobutylicum
ggaggaaaaaactatcttttaaaagtttatagtaaataaaaaaaaattattaatgtaaaaatatactaagtatagaatatttataatagggggtattaac

SEQ ID NO: 56 - DNA, 25 bp, Artificial Sequence
  Synthetic primer
taggtaccgtctggtttgcttttgc

SEQ ID NO: 57 - DNA, 24 bp, Artificial Sequence
  Synthetic primer
taagatctcatgccgggtaatacc

(19) United States
(12) Patent Application Publication
(10) Pub. No.: US 2005/0003357 A1
(43) Pub. Date: Jan. 6, 2005

(54) Novel promoters inducible by dna damaging conditions or agents and uses thereof

(76) Inventors: Jozef Anne, Winksele (BE); Sandra Nuyts, Tielt Winge (BE); Philippe Lambin, Bousval (BE)

Correspondence Address: CLARK & ELBING LLP, 101 FEDERAL STREET, BOSTON, MA, 02110, US

(21) Appl. No.: 10/479,527

(22) PCT Filed: Jun. 21, 2002
(86) PCT No.: PCT/BE02/00105

(30) Foreign Application Priority Data
Jun. 21, 2001 (GB) 0115115.8

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

The present invention relates a method of converting a promoter into a promoter which is inducible upon genotoxic compounds or conditions. The present invention further relates to a method of reducing the basal expression level of promoter which is inducible upon genotoxic compounds or conditions. These methods provides novel nucleotide sequences, vectors and host cells for the expression of proteins under the control of genotoxic conditons or compounds. The novel expression system has wide industrial applications into the field of recombinant protein production but has also clinical applications such as the controlled expression of therapeutic compounds in hypoxic tissues such as tumors.

### FIELD OF THE INVENTION

The present invention provides novel nucleotides sequences comprising promoter sequences with modified response towards DNA damaging agents and conditions. The invention also relates to vectors comprising these novel sequences and hosts transformed with these vectors. The invention further relates to the use of said modified host cells in therapeutic applications such as the treatment of cancer. The invention also relates to the use of modified nucleotide sequences, vectors and host cells for the recombinant production of proteins.

### BACKGROUND OF THE INVENTION

In the search for new therapeutic modalities for cancer, gene therapy has gained enormous interest over the last years. Many strategies to apply gene therapy have been developed and even more vectors to deliver the gene of interest have been constructed. However, one of the major pitfalls of gene therapy is still the lack of specificity of gene delivery. Developing a good gene therapy protocol involves the use of a tumour-specific vector system and gene expression limited to the tumour only. This will result in a high therapeutic index: high local tumour control with low systemic side effects.

Recently, the use of bacteria as tumour-specific protein transfer system has gained interest. Attenuated Salmonella (Pawelek, J. M. et al, 1997, Cancer Res. 54:4537-4544., Platt, J., S. Sodi et al, 2000, Eur. J. Cancer 36:2397-2402.), anaerobic Bifidobacterium (Zappe, H. et al, 1988, Appl. Environ. Microbiol. 54:1289-1292.) and apathogenic Clostridium (Fox, M. E. et al, 1996, Gene Ther. 3:173-178., Lambin, P. et al, 1998, Anaerobe 4:183-188; Lemmon, M. J. et al, 1997, Gene Ther. 4:791-765.) have shown to give selective colonisation in tumours without the presence of vegetative bacteria in the normal tissues (Lambin, ref supra). Moreover, the use of bacteria as protein transfer system is very safe since treatment can be stopped at any time by addition of the appropriate antibiotic (Theys, J. et al, 2001, FEMS Immunol. Med. Microbiol. 30:37-41.) The anaerobic gram positive bacterium Clostridium acetobutylicum was genetically engineered to express therapeutic proteins like mouse tumour necrosis factor α (mTNF-α) locally in the tumour under the control of a strong but constitutive promoter. (Theys, J. et al, 1999, Appl. Environ. Microbiol. 65:4295-4300.)

Apart from temporal and spatial expression high levels of a therapeutic protein are desired. This would solve the problems associated with systemic administration of therapeutic proteins like TNF-α where hepatotoxicity and life-threatening hypotension occur as major side-effects (Old L J. 1985, Science 230:630-636.). Limiting expression of toxic agents to the tumour cell is extremely important if damage to the surrounding normal tissues is to be avoided. Anaerobic bacteria selectively colonise the hypoxic-necrotic areas of solid tumours which are absent in healthy normal tissues and genetically engineered bacteria will secrete therapeutic proteins locally in the tumour (Theys, J. et al, 2001, Cancer Gene Ther. 8, 247-297; Theys, J. et al, 1999, Appl. Environ. Microbiol. 65:4295-4300). The use of bacterial host with a radio-inducible promoter would prevent expression in other necrotic tissues outside the tumour. In this manner, the combination of radiotherapy, one of the standard treatment modalities in cancer, and genetically engineered bacteria as tumour specific protein transfer system, enables the expression of therapeutic agents locally in the tumour due to both spatial and temporal control of protein expression.

Preferably protein expression would only occur after radiotherapy, so gene expression will be switched on and physicians will know from what time on the therapeutic protein will be present. Hallahan, D. E. et al in (1995) Nature Med. 1:786-791 describe an adenoviral vector wherein TNF-alpha is positioned under the control of the radiation inducible Egr-1 promoter.

It was earlier demonstrated that the recA promoter, belonging to the SOS-repair system of bacteria, is induced by radiotherapy, already at the clinically relevant dose of 2 Gy (Nuyts, S. et al, 2001. Anticancer Res. 21:.857-862; Nuyts, S. et al, 2001, Radiat Res. 155:716-726; Nuyts, S. et al, 2001, Gene Therapy, In press.). A single dose of 2 Gy significantly increased mTNF-α secretion by recombinant clostridia with 44%. Moreover, gene activation could be repeated with a second dose of 2 Gy, which makes it promising for clinical use, since in patient settings, daily fractions of 2 Gy are used (Nuyts, S. et al, 2001 cited supra)

All genes belonging to the SOS-repair system are activated by the presence of DNA damage. In non-activated conditions, a repressor called LexA or DinR (for Bacillus subtilis) binds on a specific operator sequence called respectively SOS-box (for Gram-negative bacteria) or Cheo box for Gram-positive bacteria. In addition to its role in homologous recombination, RecA functions as a coprotease for the LexA protein. In a healthy cell, LexA represses the expression of genes encoding DNA repair proteins (SOS genes). Upon injury of DNA, LexA catalyzes its own digestion, thereby allowing synthesis of necessary SOS proteins. However, LexA can only induce self-catalysis when activated by a ssDNA-RecA filament. A single filament will bind and activate several LexA proteins, each of which then cleaves other bound proteins. Thus, ssDNA-RecA, a product of DNA injury, stimulates DNA repair. through an increased transcription of the SOS-genes (Cheo, D. L. et al, 1991, J. Bacteriol. 173:1696-1703.; Miller, R., and T. Kokjohn, 1990, Annu. Rev. Microbiol. 44:365-394.). These genes will play a role in repairing the original DNA damage.

Both LexA and DinR bind to their operator sequence as dimers (Kim, B., and J. W. Little. 1992, Science 255:203-205., Yazawa, K. et al, 2000, Cancer Gene Ther. 7:269-274.). The consensus sequence for the Cheo box in Gram-positive bacteria is 5′ GAACNNNNGTTC 3′ (cheo et al cited supra). This consensus sequence is positioned within promoter regions such that the regulatory molecule LexA bound at these sites could interfere with the initiation of transcription by RNA polymerase. Several genes can be found which have 2 or more putative Cheo boxes and for those, in which repressor binding is proven, the distance between the two boxes is 15 to 16 bp (yazawa cited supra ).

A system similar as described for Clostridium is known for gram negative bacteria such as E. coli. When cells like E. coli are subject to excessive DNA damage, a system (the SOS response) that stops DNA synthesis and invokes massive DNA repair is triggered. The SOS system is regulated by RecA. If there is any DNA damage present during replication, RecA will associate with the single stranded DNA that is generated after DNA damage. RecA will also associate with a protein called LexA. LexA is a repressor that normally turns off a large group of genes associated with DNA repair, including recA, uvrA, uvrB and uvrD. Each of these genes has a similar consensus sequence called the SOS BOX (5′-CTGNNNNNNNNNNCAG-3′, where N can be any base). LexA binds to the SOS box, turning off genes with an SOS box in their promoters. However, when RecA interacts with single stranded DNA, RecA is “activated” such that RecA binds to LexA. LexA bound to RecA does not bind to the SOS box, and thus all the genes with an SOS box (mainly DNA repair genes) are turned on. The controlling factor in this system is the presence of single stranded DNA. Some genes with SOS boxes inhibit cell division. Thus when the LexA-RecA complex is formed, DNA repair is initiated and cell division is inhibited. When the damaged DNA is repaired, there will be no means to activate the RecA such that it binds to LexA, and thus LexA will again inhibit all genes with SOS boxes and related DNA repair will cease and cell division will continue.

Despite the wide knowledge on DNA damage mediated expression of proteins and despite the variety of expression systems for proteins by anaerobic organisms in hypoxic tissues, there is still a need for DNA constructs, vectors and host cells which allow inducible expression with low levels of basal expression. There is also a need for DNA constructs, vectors and host cells which allow more regulated and higher expression of proteins than those known in the art.

### SUMMARY OF THE INVENTION

The present invention relates to an isolated and purified polynucleotide comprising at least one first sequence element inserted in a second sequence element wherein the first sequence element is a repressor binding element of a promoter which is inducible by DNA damaging agents or conditions and wherein the second sequence element is a promoter sequence. The promoter can be not inducible by a DNA damaging agent or condition but also can be inducible by a DNA damaging agent or condition. The polynucleotide can be positioned 5′ to a nucleotide sequence suitable for the introduction of a third sequence element.

The invention relates to a method of converting a promoter which is not inducible by DNA damaging agents or conditions into a promoter which is inducible by radiation, genotoxic compounds or DNA damaging compounds comprising the step of inserting at least one repressor binding element of a promoter which is inducible by a DNA damaging compound or condition into said non inducible promoter.

The invention relates to a method of increasing the induction level of a first promoter which is inducible by genotoxic compounds or conditions comprising the step ofinserting at least one repressor binding element of a said first promoter or a second promoter which is inducible by a DNA damaging compound or condition into the first inducible promoter.

The invention relates to a method of decreasing the basal expression level of a first promoter which is inducible by genotoxic compounds or conditions comprising the step of inserting at least one repressor binding element of a said first promoter or a second promoter which is inducible by a DNA damaging compound or condition into the first inducible promoter.

The invention further relates to a vector comprising a nucleotide sequence of the present invention.

The invention further relates to a bacterial host cell transfected with the vectors of the present invention.

The invention further relates to a pharmaceutical composition comprising a cell of the present invention in admixture with at least one pharmaceutically acceptable carrier.

The invention further relates to a method of expressing a therapeutic protein or a protein converting a precursor into a therapeutic compound comprising a first step of administering to an individual of the pharmaceutical composition and a second step of subjecting the person to a DNA damaging condition and/or administering to an individual a DNA damaging compound or a precursor thereof.

The invention also relates to a method for the in vitro production of recombinant proteins comprising the step of contacting a culture of host cells of the present invention with a DNA damaging compound or condition.

### BRIEF DESCRIPTION OF THE FIGURES

FIG. 1 shows the releative increase of mTNF-α secretion in Clostridium acetobutylicum DSM792 pIMP-recA-mTNF-α without Cheo box (filled boxes) and Clostridium acetobutylicum DSM792 pIMP-recA-mTNF-α with an extra Cheo box (empty boxes) after a single dose of 2 Gy in function of time after irradiation. The bars represent data from three independent experiments.

FIG. 2 shows the relative increase of mTNF-α secretion in Clostridium acetobutylicum DSM792 pIMP-eglA-mTNF-α (filled boxes) and Clostridium acetobutylicum DSM792 pIMP-eglA-mTNF-α with a Cheo box (empty boxes) after a single dose of 2 Gy in function of time after irradiation. The bars represent data from three independent experiments.

FIG. 3 presents the results of RT-PCR on irradiated and non-irradiated RNA extracted from Clostridium acetobutylicum DSM792. The upper panel represents the amplification of a 650 bp internal fragment of 16S rRNA which functions as an internal standard to ensure equal amounts of RNA were used in each reverse transcription reaction. The lower panel represents the amplification of a 470 bp internal fragment of mTNF-α.A reference DNA-ladder is shown on the right.RNA extracted from C. acetobutylicum DSM792 transformed with pIMP-eglACheo-mTNF-α is shown in lanes 1 and 2; pIMP-recA-mTNF-α is shown in lanes 3 and 4; pIMP-recAextraCheo-mTNF-α is shown in lanes 5 and 6; pIMP-eglA-mTNF-α is shown in lanes 8 and 9; pIMP-recAdeletedCheo-mTNF-α is shown in lanes 10 and 11; Lane 7 shows a positive control for 16S rRNA (PCR performed on chromosomal DNA C. acetobutylicum)

FIG. 4 shows the activity of an expressed luciferase reporter gene operably linked to a recA promoter after (E=exposed; squares) or without irradiation (NE=non exposed, diamonds) (RLU=relative light units)

FIG. 5 shows the induction factor of a luciferase gene operably linked to a RecA promotor. The induction factor is the ratio between the expression level under inducing conditions and the expression level under non inducing conditions.

### DEFINITIONS

“consensus sequence” in the present invention refers to a representation of a sequence alignment of related sequences of repressor binding elements wherein in this representation the most frequently occurring residue at a certain position is shown. Therefore, the consensus sequence represents the consensus sequence and also the naturally occurring variations on the consensus sequence as represented by the individual sequences of the alignment, and also engineered sequences where one or more residues are modified with respect to the consensus sequence, said engineered sequence still being able to bind the repressor.

“Expression” as used herein, refers to the transcription and translation to gene product from a polynucleotide and/or a full-length gene coding for the sequence of the gene product. In the expression, a DNA chain coding for the sequence of a gene product is first transcribed to a complementary RNA which is often a messenger RNA and, then, the thus transcribed messenger RNA is translated into the above-mentioned gene product if the gene product is a protein.

“Gene products” as used herein, refers to any molecule capable of being encoded by a nucleic acid, but not limited to, a polypeptide or another nucleic acid, e.g. DNA, RNA, dsRNA, ribozyme, DNAzyme etc. The term “Gene product” may thus refer to a polypeptide produced by transcription of a specific DNA coding region into mRNA followed by translation of the mRNA by a ribosome. Such a polypeptide may also refer to as a “protein”. The polynucleotide, which encodes for the gene product of interest, is not limited to naturally occurring full-length “gene” having non-coding regulatory elements.

“promoter” and “promoter region” as used herein, refer to a sequence of DNA, usually upstream of the gene product coding sequence, which controls the expression of the coding region by providing the recognition for RNA polymerase and/or other factors required for transcription to start at the correct site. Promoter sequences are necessary but not always sufficient to drive the expression of the gen.

The term “strong promoter” as used herein refers to a promoter operably linked to an encoding polynucleotide sequence that results in high levels of expression and/or expression independent of cell cycle.

“DNA damaging compound (synonym: genotoxic compound) or DNA damaging agent” as used herein refers to molecules which damage or modify the backbone/and or side chains resulting in the generation of single stranded DNA fragments.

“DNA damaging conditions” as used herein relate to conditions such as high energy radiation, e.g. UV radiation, gamma or X ray radiation which modify or damage the backbone/and or side chains of DNA resulting in the generation of single stranded DNA fragments.

“Gram negative” refers to the inability of a bacterium to resist decolorisation with alcohol after being treated with Gram crystal violet. Optionally, these bacteria can be counterstained with safranin, imparting a pink or red colour to the bacterium when viewed by light microscopy

“Gram positive” refers to ability of a bacterium to resist decolorisation with alcohol after being treated with Gram crystal violet, imparting a violet colour to the bacterium when viewed by light microscopy.

The term “strong promoter inducible by DNA damaging conditions or compounds” used herein refers to naturally occurring promoters such as the recA promoter. It also refers to strong constitutive promoters or promoters that in their nature are not inducible by radiation, by genotoxic compounds or by DNA damaging agents, but which after insertion of one or more repressor binding elements in said promoter region have been made inducible by radiation, by genotoxic compounds or by DNA damaging agents.

“Inducible promoter” as used herein, refers to a promoter which can be activated by addition of a particular molecule or a particular agent or by exposing to physical conditions such as irradiation, called an inducer.

“Operably linked” as used herein, refers to a state of joinder of a promoter and a full length gene or an encoding polynucleotide, wherein RNA polymerases are capable of recognising the promoter.

“repressor-binding element” as used herein refers a specific operator sequence in the promoter sequence of gram positive or gram negative bacteria. For example, repressor binding elements of gram-positive bacteria to which the repressor dinR binds upon DNA damage are sequences with the Cheo box consensus sequence GAACNNNNGTTC [SEQ ID NO 1] or the DinR box consensus sequences CGAACRNRYGTTYC [SEQ ID NO 2] Examples of such sequences are shown in table 1. For example, repressor binding elements of gram-negative bacteria to which the repressor LexA binds upon DNA damage are sequences with the SOS box consensus sequence CTGNNNNNNNNNNCAG [SEQ ID NO 3]. Examples of such sequences are shown in table 2.

“therapeutic protein” as used herein relates to any protein used in the treatment of a mammalian disease. Where the disease involves pathogenic tissues such as cancer tumours or bacterial infection, this term can refer to proteins which have a toxic, cytotoxic or cytostatic effect or can refer to proteins which convert a molecule into a molecule with cytostatic or cytotoxic effects (e.g. prodrug). Therapeutic proteins as used herein related to the treatment of an ischemic disease can also relate to growth factors, angiogenic or arteriogenic factors (e.g. PIGF or VEGF or other fam0ily members). Therapeutic proteins as used herein can relate to drug delivery can refer to a drug or a pore opening protein, for example.

### DETAILED DESCRIPTION OF THE INVENTION

The present invention is based on the surprising finding that the introduction of a repressor binding elements such as the Cheo box in a constitutive promoter such as the eglA promoter causes the modified promoter to respond to ionising radiation in contrast to the unmodified eglA promoter. The Cheo box was introduced about 70 bp upstream of the ribosome binding site to make sure interaction could occur between RNA polymerase and the promoter which could inhibit transcription. This positioning is within the range of between about −42 and about −106 reviewed by Yazawa et al (cited supra).

The present invention is further based on the finding that a repressor element such as the Cheo box in a promoter such as the recA promoter of a C.acetobutylicum DSM792 is a necessary and sufficient element responsible for induction after action of a DNA damaging condition or compound, e.g. ionising irradiation. After deletion of the Cheo box there was no increase in protein expression after irradiation in contrast with the ‘wild-type’ recA promoter where radiation-induced expression was present. Incorporation of a second Cheo box of about 50 bp upstream of the first, increased radio induced expression from about a 40% the expressed protein for the ‘wild-type’ promoter to about 400% for the mutated promoter, when compared to the non-irradiated conditions.

A first embodiment of the invention refers to the insertion of one or more sequence elements (first sequence elements) into nucleotide sequences of promoters (second sequence elements) which are not inducible by DNA damaging agents or conditions, said insertion or insertions leading to a modified promoter which becomes inducible upon action of a DNA damaging agent or condition.

A second embodiment of the invention refers to the insertion of one or more sequence elements (first sequence elements) into nucleotide sequences of promoters (second sequence elements) which are already inducible by DNA damaging agents or conditions, said insertion or insertions leading to a modified promoter which remains inducible upon DNA damaging agents or conditions but results in an increased expression level of protein compared to the expression level of the promoter before the insertion of one or more first sequence elements.

In one aspect of this embodiment the one or more sequence elements being introduced are repressor binding elements of promoters of genes which are switched on in the presence of DNA damaging compounds or conditions. These genes are mostly involved in the DNA damaging repair processes of living cells and occur in a wide range of organisms including higher eukaryotes such mammals and vascular plants, but also lower eukaryotes such as insects and nematodes and fungi, such as fission and budding yeast and in prokaryotes, including both gram positive and gram negative bacteria. Especially lower organism which live in high environmental stress (such as light, radiation, and toxic environments) are adapted to respond to these stress conditions by a sophisticated DNA repair system. Promoters which are inducible after DNA damaging conditions or compounds are well characterised in both gram negative and gram positive bacteria.

In one aspect of the invention, the repressor binding element which is introduced into a promoter can be any element when said repressor binding element is being recognised by a repressor in the host in which the modified promoter was introduced. The recognition can be evaluated by contacting the repressor with a putative repressor binding oligonucleotide. This binding can be assayed for example with a gel retardation assay, also known as “bandshift”, wherein a labelled nucleotide shows a retarded migration to a gel when a protein is bound to said labelled nucleotide.

In a more preferred aspect of this embodiment the repressor binding elements are bacterial repressor binding elements of promoters activated by DNA damaging conditions or compounds. These are known in gram positive bacteria as Cheo boxes and DinR boxes. The different sequences of Cheo boxes are represented by the Cheo box consensus site of 12 nucleotides GAACNNNNGTTC [SEQ ID NO 1] or alternatively by the DinR consensus site of 14 nucleotides CGAACRNRYGTTYC [SEQ ID NO 2]. A list of examples of repressor binding elements comprising Cheo boxes, without the intention to restrict the scope of the invention only to these examples, is shown in Table 1.

TABLE 1

Repressor binding sequences of RecA like
sequences from different gram positive bacteria

| | | sequence repressor | |
|---|---|---|---|
| species | promoter | binding element | SEQ ID number |
| Bacillus | rec A (−51) | CGAATATGCGTTCG | SEQ ID NO 4 |
| subtilis | dinA | CGAACTTTAGTTCG | SEQ ID NO 5 |
| | dinB | AGAACTCATGTTCG | SEQ ID NO 6 |
| | dinC (−24) | CGAACGTATGTTTG | SEQ ID NO 7 |
| | dinC (−53) | AGAACAAGTGTTCG | SEQ ID NO 8 |
| | dinR (−39) | CGAACCTATGTTTG | SEQ ID NO 9 |
| | dinR (−67) | CGAACAAACGTTTC | SEQ ID NO 10 |
| | dinR (−104) | GGAATGTTTGTTCG | SEQ ID NO 11 |
| Bacteroides | recA (−20) | CGAATTAAACTTTG | SEQ ID NO 12 |
| fragilis | recA (−107) CGAACGGATCATCG | SEQ ID NO 13 | |
| Clostridium | recA | AGAACTTATGTTCG | SEQ ID NO 14 |
| perfringens | | | |
| Corynebacterium | recA | CGTAGGAATTTTCG | SEQ ID NO 15 |
| glutamicum | | | |
| Corynebacterium | recA | AGAATGGTCGTTAG | SEQ ID NO 16 |
| pseudotuberculosis | | | |
| Deinococcus | recA | CGATCCTGCGTAAG | SEQ ID NO 17 |
| radiodurans | | | |
| Mycobacterium | recA | CGAACAGATGTTCG | SEQ ID NO 18 |
| leprae | | | |
| Mycobacterium | recA | CGAACAGGTGTTCG | SEQ ID NO 19 |
| smegmatis | | | |
| Mycobacterium | recA | TCGAACAGGTGTTCGA | SEQ ID NO 20 |
| tuberculosis | lexA | TCGAACACATGTTTGA | SEQ ID NO 21 |
| Staphylococcus | recA | CGAACAAATATTCG | SEQ ID NO 22 |
| aureus | | | |
| Streptococcus | recA | CGAACATGCCCTTG | SEQ ID NO 23 |
| mutans | | | |
| Streptomyces | recA | CGAACATCCATTCT | SEQ ID NO 24 |
| lividans | | | |
| Thermotoga | recA | CGAATGTCAGTTTG | SEQ ID NO 25 |
| maritima | | | |

In another more preferred aspect of this invention the repressor binding elements are from gram negative bacteria. In these organism the element is known as the SOS box. The different sequences of SOS boxes are represented by the SOS box consensus site of 16 nucleotides CTGNNNNNNNNNNCAG [SEQ ID NO 3]. A list of examples representing SOS boxes, without the intention to restrict the scope of the invention only to these examples, is shown in Table 2.

TABLE 2

sos boxes of promoters from
the gram negative bacterium E. coli.

| | sos | |
|---|---|---|
| sos box sequence | box name | SEQ ID |
| tactgtatgagcatacagta | recA | SEQ ID NO 26 |
| tactgtatattcattcaggt | uvrA | SEQ ID NO 27 |
| aactgtttttttatccagta | uvrB | SEQ ID NO 28 |
| tactgtacatccatacagta | sulA | SEQ ID NO 29 |
| atctgtatatatacccagct | uvrD | SEQ ID NO 30 |
| tactgtataaataaacagtt | mucAB | SEQ ID NO 31 |
| tactgtgtatatatacagta | clo13 | SEQ ID NO 32 |
| tgctgtatatactcacagca | lexA-1 | SEQ ID NO 33 |
| aactgtatatacacccaggg | lexA-2 | SEQ ID NO 34 |
| tgctgtatataaaaccagtg | cle1-1 | SEQ ID NO 35 |
| cagtggttatatgtacagta | cle1-2 | SEQ ID NO 36 |
| tactgtatatgtatccatat | Co11b | SEQ ID NO 37 |
| tactgtatataaacacatgt | Co1A-1 | SEQ ID NO 38 |
| acatgtgaatatatacagtt | Co1A-2 | SEQ ID NO 39 |
| atctgtacataaaaccagtg | Co1E2 | SEQ ID NO 40 |
| tactgtatataaaaacagta | umuDC | SEQ ID NO 41 |
| tactgtatataaaaccagtt | recN-1 | SEQ ID NO 42 |
| tactgtacacaataacagta | recN-2 | SEQ ID NO 43 |

In one embodiment the insertion of one or more repressor binding elements occurs between 1 and 1000 basepairs upstream from the ribosome binding element of the second sequence element, alternatively occurs between 1 and 200 base pairs, or occurs between 46 and 104 basepairs upstream from said ribosome binding element.

Another aspect of this embodiment relates to non inducible promoters which are modified by the insertion of one or more repressor binding elements. These promoters can be weak promoters but are preferably strong promoters. A candidate promoter can be evaluated by any reporter assay wherein a promoter is operably linked to a reported gene and where, after introduction in the appropriate host, the amount or activity of the reporter gene is assayed. Examples of such reporter genes or luciferase or chloramphenicol transferase. A preferred promoter according the present invention is the EglA promoter of Clostridium.

Another aspect of the invention relates to inducible promoters which are modified by the insertion of one or more repressor binding elements. These promoters can be any promoter which is inducible by DNA damaging compounds or conditions. Examples of such promoters in gram negative bacteria are promoters to which the repressor LexA can bind and are mentioned in table 2. Examples of such promoters in gram positive bacteria are promoters to which the repressor DinR can bind are mentioned in table 1.

Another aspect of the present invention are vectors comprising the modified sequences which are inducible upon DNA damaging compound or conditions. Said vectors contain a nucleotide sequence located 5′ to the promoter (known as cloning site or multiple cloning site) for operably linking to the promoter a sequence which is described according to this invention as a third sequence element. This third sequence element is the gene which will be transcribed and translated after action by DNA damaging conditions or compounds. Further, vectors can optionally comprise an additional nucleotide sequence which results in the expression of a fusion protein with as a first part of the fusion protein the protein being expressed from the third sequence element and as a second part a signal peptide, a tag for the recognition of an antibody (e.g. myc, Flag, HA tag), a tag for the recognition of a protease (e.g. thrombin, Factor X, Enterokinase site), a protein with enzymatic activity or a protein or peptide which is able to bind to a carrier (e.g. GST, maltose binding protein, His tag). A preferred version of fusion protein is a protein fused to a peptide which allows the secretion of the protein. Preferred examples of such signal peptides (and accompanying promoter) are for Clostridium the eglA isolated from Clostridium acetobutylicum, clostripain promoter and signal peptide from Clostridium histolyticum, glutamine synthethase from Clostridium beijerinckii

Another aspect of this invention relates to host cells transformed with the vectors of the present invention. These host cells are preferably bacteria. The bacteria are preferably bacteria which have a certain tissue or organ specific preference.

In the embodiments related to the treatment of an hypoxic tissue, the bacteria preferably are non pathogenic bacteria and even more preferably anaerobic or facultative anaerobic non pathogenic bacteria; in a most preferable embodiment they are anaerobic non pathogenic bacteria; in a preferred embodiment these non pathogenic gram positive anaerobic bacteria are bacteria of the species Clostridium. Examples of gram-positive bacteria thereof are Clostridium acetobutylicum, Clostridium sporogenes, Clostridium beijerinckii, Clostridium oncolyticum, Clostridium butyricum, Clostridium novyi as well as Bifidobacterium infantis, Bifidobacterium bifidum, Bifidobacterium longum

Other examples of Gram-positive bacteria useful for different applications are examples of aerobic or facultative aerobic gram positive bacteria (for in vitro expression) Bacillus subtilis, Streptomyces lividans, Streptomyces coelocolor, Lactobacillus spp., Lactococcus spp.

Examples of gram negative bacteria are Salmonella typhimurium, but also other species such as other intracellular bacteria or species such as E. coli, Pseudomonas, Rhizobium.

Another aspect of this invention relates to pharmaceutical compositions and methods of treating patients with bacterial host cells transfected with the nucleotides of the present invention. Preferably, the treatment according to the present invention relates to the treatment of cancer tissue and more preferably to the treatment of cancer tissue subject to hypoxic conditions. The treatment however also relates to the treatment of other disorders with hypoxic conditions such as abscesses and ischemic tissues. Recently, the use of bacteria as tumour-specific protein transfer system has gained interest. Attenuated Salmonella (Pawelek cited supra, Platt cited supra.), anaerobic Bifidobacterium (Zappe et al cited supra) and apathogenic Clostridium (Fox et al, cited supra, Lambin et al, 1998 cited supra; Lemmon et al cited supra) have shown to give selective colonisation in tumours without the presence of vegetative bacteria in the normal tissues (Lambin, ref supra).

The treatment according to the present invention has the advantage that the action of irradiation or a administration of a genotoxic compound, in addition to its curative effect, acts as an inducer of a gene which expresses a therapeutic protein. As an alternative, genotoxic drugs can be administered at a lower dosage which results in limited systemic side effects. The genotoxic compound will, however, act locally in the host cell as an inducer of the gene encoding a therapeutic protein.

The treatment of tumours according to the present invention relates to tumours occurring in mammals and in particular to humans. Examples of tumours are sarcomas, carcinomas, or other solid tumor cancers include, but are not limited to, germ line tumors, tumors of the central nervous system, breast cancer, prostate cancer, cervical cancer, renal cancer, bladder cancer, uterine cancer, lung cancer, ovarian cancer, testicular cancer, thyroid cancer, mesoendothelioma, mesothelioma, astrocytoma, glioma, pancreatic cancer, stomach cancer, liver cancer, colon cancer, and melanoma.

In accordance with the present invention the treatment comprises subjecting an individual to host cells of the present invention, the host cells having a vector comprising inducible promoter, inducible upon action of a DNA damaging condition or compound. The DNA damaging condition can be irradiation with high energy radiation such as beta rays, gamma rays or X-rays. The treatment may comprise a single dose of irradiation or may comprise several doses of irradiation (fractionated doses). The effective dose of irradiation can be calculated using methods known in the art taking into account the overall health of the patient and the type and location of the solid tumour. An illustrative example of a course of radiation treatment for a human patient with a solid tumour is local administration of irradiation to the tumour site of 2 Gy/day for 5 days per week for 6 weeks (total exposure of 60 Gy).

Alternatively for the treatment of tissues which are located at the outside of the body or which are easily accessible, treatment with ultraviolet radiation or string light sources can be used.

The vectors and the host cells of the present invention allow the induction of a gene by a compound with genotoxic properties which is being used as such in cancer chemotherapy or in a combination treatment of chemotherapy and irradiation. Examples of such compounds with genotoxic properties are mitomycin, alkylating agents, antimetabolites, bioreductive drugs.

The present invention allows the inducible expression of genes at a specific part of the body. The specific part of the body may be determined by a tissue state such as hypoxia, or by a particular preference for a part of the body by the host bacterial cell. In accordance with the definitions of the present invention the genes being encoded are transcribed and translated from what is described as the third sequence element. This allows the use of genes which are normally toxic for healthy cells when administered to an individual, and allows the use of genes which are capable of converting locally a harmless precursor compound into a toxic compound, thereby resulting into the time and place dependent activity of a anticancer agent. Proteins with toxic or cytotoxic activities and protein which convert a non toxic compound (prodrug converting enzymes) into a toxic compound are defined in the context of cancer treatment as therapeutic proteins.

Examples of cytotoxic proteins are saporin, ricins, abrin and ribosome inactiviting proteins (RIPs), Pseudomonas exotoxin, inhibitors of DNA, RNA or protein synthesis, DNA or RNA cleaving molecules such as DNase and ribonuclease, proteases, lipases, phospholipase), prodrug converting enzymes (e. g., thymidine kinase from HSV and bacterial cytosine deaminase), light-activated porphyrin, ricin, ricin A chain, maize RIP, gelonin, E. coli cytotoxic necrotic factor-1, Vibrio fischeri cytotoxic necrotic factor-1, cytotoxic necrotic factor-2, Pasteurella multicida toxin (PMT), cytolethal distending toxin, hemolysin, verotoxin, diphtheria toxin, diphtheria toxin A chain, trichosanthin, tritin, pokeweed antiviral protein (PAP), mirabilis antiviral protein (MAP), Dianthins 32 and 30, abrin, monodrin, bryodin, shiga, a catalytic inhibitor of protein biosynthesis from cucumber seeds (see, e. g., International Publication WO 93/24620), Pseudomonas exotoxin, E. coli heat-labile toxin, E. coli heat-stable toxin, EaggEC stable toxin-1 (EAST), biologically active fragments of cytotoxins and others known to those of skill in the art. See, e. g. O'Brian and Holmes, Neidhardt et al. (eds.), pp. 2788-2802, ASMPress, Washington, D. C.) Yet other exemplary gene products of interest include, but are not limited to, methionase, aspariginase and glycosidase.

Other therapeutic proteins can be antiangiogenic factors, such as endostatin; angiostatin; apomigren; anti-angiogenic antithrombin III; proteolytic fragments of fibronectin; uPA receptor antagonist; I6 kDa proteolytic fragment of prolactin; t 7.8 kDa proteolytic fragment of platelet factor-4; anti-angiogenic 13 amino acid fragment of platelet factor-4; antiangiogenic 14 amino acid fragment of collagen I; anti-angiogenic 19 amino acid peptide fragment of Thrombospondin I; anti-angiogenic 20 amino acid peptide fragment of SPARC, RGD and NGR containing peptides; small anti-angiogenic peptides of laminin, fibronectin, procollagen and EGF, and peptide antagonists of integrin av 3 and the VEGF receptor; can also be a Flt-3 ligand.

Other therapeutic proteins in accordance with the present invention are cytokines which result in a significant antitumor immune response. Examples are IL-1; IL-2; IL4; IL-5; IL-15; IL-18; IL-12; IL-10; GM-CSF; INF-y; INF-a; SLC; EMAP2; MIP-3a; MIP-3; an MHC gene such as HLA-B7; members of the TNF family, including but not limited to tumor necrosis factor-a (TNF-a), tumor necrosis factor-P (TNF-P), (TRAIL), (TRANCE); CD40 ligand (CD40L); LT-a; LT-P; OX40L; CD40L; FasL; CD27L; CD30L; 4-1BBL; APRIL; LIGHT; TL1; TNFSF16, TNFSF17, and AITR-L.

Examples of therapeutic pro-drug converting enzymes are HSVTK (herpes simplex virus thymidine kinase) and VZVTK (varicella zoster virus thymidine kinase), which selectively phosphorylate certain purine arabinosides and substituted pyrimidine compounds, converting these compounds to metabolites that are cytotoxic or cytostatic. For example, exposure of the drug ganciclovir, acyclovir, or any of their analogues (e. g., FIAU, FIAC, DHPG) to cells expressing HSVTK allows conversion of the drug into its corresponding active nucleotide triphosphate form; E. coli guanine phosphoribosyl transferase, converting thioxanthine into toxic thioxanthine monophosphate; alkaline phosphatase, converting inactive phosphorylated compounds such as mitomycin phosphate and doxorubicin-phosphate to toxic dephosphorylated compounds; fungal (eg Fusarium oxysporum) or bacterial cytosine deaminase, which converts 5-fluorocytosine to the toxic compound 5-fluorouracil; carboxypeptidase G2, cleaving glutamic acid from para-N-bis (2-chloroethyl) aminobenzoyl glutamic acid, thereby creating a toxic benzoic acid mustard; Penicillin-V amidase, which converts phenoxyacetabide derivatives of doxorubicin and melphalan to toxic compounds Moreover, a wide variety of Herpesviridae thymidine kinases, including both primate and non-primate herpesviruses, are suitable, including Herpes Simplex Virus Type 1 Herpes Simplex Virus Type 2 Varicella Zoster Virus.

Other therapeutic compounds are bacterial proteins, which upon expression in mammalian cells perform a cytotoxic function. Examples hereof are colicin, such as colicin E3 , V, A, E1, E2, Ia, Ib, K, L, ; cloacin, such as cloacin DF13; pesticin A1122; staphylococcin 1580; butyricin 7423; vibriocin pyocin RI or AP41; megacin A-216 and BRP (Bacteriocin Release Protein) from Enterococus cloacae.

Other therapeutic proteins for use according to the present invention are pore opening compounds such as the bacterial toxin Zot from Vibrio cholerae, which act as pore forming molecules, thereby facilitating the transport of pharmaceutical small molecules which are normally not able to penetrate the membrane of a mammalian or human cell.

Other therapeutic proteins according the present inventions are vascularisation promoters or growth factors with angiogenic and/or arteriogenic properties such as members of the VEGF family (PLGF, VEGF) for the treatment of ischemic disease.

In another embodiment of the present invention, a transformed bacterium, preferably with no or little toxic side effects, and preferably with a tissue or organ specific preference, is introduced into a patient wherein a persistent bacterial infection occurs (eg rectum, bladder, intestine, stomach). Upon induction with a genotoxic compound or a DNA damaging condition the host bacterium will express a toxic protein or converting a pro-drug in to drug toxic compound resulting in the killing of the persistent bacteria. The introduced host cell can, if desired, be killed with an appropriate antibiotic.

Several methods are known to deliver the bacterial host cell to an individual being treated according to this invention. Methods of introduction include but are not limited to intradermal, intrathecal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e. g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e. g., by use of an inhaler or nebulizer, and formulation with an aerosolizing agent. In a specific embodiment, it may be desirable to administer the pharmaceutical compositions of the invention locally to the area in need of treatment; this may be achieved by, for example, and not by way of limitation, local infusion during surgery, by injection, by means of a catheter, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the host cell of the present invention can be delivered in a controlled release system. In one embodiment, a pump may be used. In another embodiment, polymeric materials can be used (see Medical Applications of Controlled Release, Langer and Wise (eds.), CRC Pres., Boca Raton, Fla. (1974) In yet another embodiment, a controlled release system can be placed in proximity of the therapeutic target, thus requiring only a fraction of the systemic dose. Other controlled release systems are discussed in the review by Langer (1990) Science 249, 1527-1533

The pharmaceutical compositions of the present invention comprise a host cell and at least one pharmaceutically acceptable carrier. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration. In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Another embodiment of the present invention is the use of the modified nucleotide sequences vectors and host cells of the present invention for the expression of genes with tight repression under basal i.e. non inducing conditions. As is shown in the present invention, the introduction of additional repressor responsive elements not only gives an increase in expressed protein but also results in a lower transcription rate under non inducing conditions. RT-PCR demonstrated that the increase in secretion was the result of increased promoter activity since higher concentrations of mRNA were present in the irradiated samples. Increased secretion of therapeutic proteins like mTNF-α in Clostridium after irradiation is thus the result of increased activity at transcriptional level. RT-PCR also demonstrated that in non-irradiated conditions, (resembling basal conditions) the addition of a Cheo box resulted in lower transcription and the deletion of a Cheo box in higher transcription. These results prove that the Cheo box functions as a repressor binding site which becomes free after DNA damage, caused by, for example, ionising irradiation, leading to a removal of repression and increased transcription.

Also genes with wild type promoters which are inducible by DNA damaging conditions or compounds can be modified by the addition of additional repressor binding elements from promoters which are inducible by DNA damaging compounds or conditions, thereby further decreasing the basal expression level of the genes which are operably linked to this modified promoter when compared to the basal expression level of the unmodified promoter.

This feature has several advantages for expression systems in general. For the expression of genes for proteins which are toxic to the host, the expression level under basal conditions has to be limited. Also for the expression of genes which are introduced into host cells for therapeutic agents the proteins preferably are not expressed under non inducing conditions.

Another embodiment relates to a novel in vitro expression system wherein hosts transfected with the vectors comprising the novel sequences of the present invention, wherein the protein expression is induced by DNA damaging conditions or compounds. The in vitro expression system of the present can be used for the expression of any protein which is a likely candidate to be expressed in bacterial cell. This expression system has desirable features such bulk growth of the host cells, inexpensive media for the growth of such host cells. In an embodiment of the present invention bacteria are exposed to radiation after a growth phase. For example, bacteria are exposed to 2 Gy with a ⁶⁰Cobalt unit at a dose-rate of 0.9 Gy/min for a time period ranging from 1 to 120 minutes or 30 to 90 minutes.

In a preferred embodiment of the expression system the induction is performed with UV irradiation. More preferably an incubator setting is used wherein the medium with host cells is transported along a UV lamp with a wavelength of 254 in order for a host cell to be subjected on average for about between 10 and 30 seconds, between 20 and 40 seconds, between 45 and 90 seconds, the distance between the host cell and the UV source is between 1 and 5 cm or between 8 and 15 cm.

The introduction of repressor binding elements into a bacterial promoter in order to obtain higher expression, preferably with less basal expression can be applied to modify any expression system which is known the person skilled in the art (for example, see Sambrook et al. cite supra, and vectors from commercial suppliers such as Novagen, Pharmacia, Gibco, Invitrogen, Stratagene)

Examples of constitutive promoters in E. coli which can be modified according to the present invention are the bla promoter from β-lactamase), the Tn5 promoter of the neomycin resistance gene, the cat promoter of the chloramphenicol resistance gene, the tet promoter of the tetracycline resistance gene, the strong constitutive EM7 and TRNA promoters.

### EXAMPLE 1

### Bacterial Strains, Plasmids and Culture Conditions

Clostridium acetobutylicum DSM792 was grown in 2×YT (Yeast Tryptone) medium at 37° C. in an anaerobic system (model 1024; Forma Scientific, Marietta, Ohio) with 90% N₂ and 10% H₂ and palladium as catalyst (Oultram et al. 1988, FEMS Micriobiol. Lett. 56, 83-88.

For primary vector construction, Escherichia coli TG1 was used (Sambrook et al cited supra). This strain was grown in Luria-Bertani (hereafter abbreviated as LB) broth at 37° C. E. coli strain ER2275 was used for in vivo methylation of plasmid DNA prior to electroporation of clostridia (Mermelstein, L. D., and E. T. Papoutsakis. 1993. Appl. Environ. Microbiol. 59:1077-1081.; Mermelstein, L. D. et al, 1992. BioTechnology 10:190-195.). The E.coli/Clostridium shuttle plasmid pIMP1 was used as cloning vector (Mermelstein, L. D. et al, 1992. BioTechnology 10:190-195.).

The murine tumour necrosis factor alpha (hereafter abbreviated as mTNF-α) cDNA was available on plasmid pIG2mTNF (obtained from Innogenetics, Gent, Belgium). Plasmid pHZ117, containing the eglA gene of C. acetobutylicum P262, was a obtained from H. Zappe (cited supra). The eglA promoter and signal sequence were used to express and secrete mTNF-α. This chimeric gene construct was present on the shuttle plasmid pIMP1, resulting in pIMP-eglA-mTNF-α (Theys, J. et al, 1999, Appl. Environ. Microbiol. 65:4295-4300.). In this plasmid, the eglA promoter was replaced by the C. acetobutylicum recA promoter, resulting in pIMP-recA-mTNF-α (Nuyts S. et al. 2001 Applied & Environmental Microbiology 67: 4464-4470.). Table 3 gives an overview of the plasmids used in this study. The recA promoter was isolated from chromosomal DNA as previously described (Nuyts, S. et al, 2001, Radiat Res. 155:716-726.). Media were supplemented, when applicable, with erythromycin (25 μg/ml) or ampicillin (50 μg/ml).

TABLE 3
characteristics of engineered plasmids

| | Derived | | | |
|---|---|---|---|---|
| | from | | Signal | Therapeutic |
| Name | plasmid | Promoter | sequence | gene |
| pIMP-eglA- | pIMP1 | eglA | eglA | mTNF-α |
| mTNF-α | | | | |
| pIMP- | pIMP1 | eglA with | eglA | mTNF-α |
| eglACheo- | | incorporated | | |
| mTNF-α | | Cheo box | | |
| pIMP-recA- | pIMP1 | recA | eglA | mTNF-α |
| mTNF-α | | | | |
| pIMP- | pIMP1 | recA with | eglA | mTNF-α |
| recAextraCheo- | | extra Cheo | | |
| mTNF-α | | box | | |
| | | incorporated | | |
| pIMP- | pIMP1 | recA with | eglA | mTNF-α |
| recAdeletedCheo- | | Cheo box | | |
| mTNF-α | | deleted | | |

### EXAMPLE 2

### Mutation of the recA and eglA Promoter, DNA Manipulations and Transformation Procedures

Introduction and/or deletion of the Cheo box in the recA and eglA promoter was done using “Quickchange Site-directed Mutagenesis kit” (Stratagene). Table 4 represents the sequences of the ‘wild-type’ recA and eglA promoters at the 3′ region. All mutations were introduced in the shuttle vectors pIMP1 containing the eglA or recA promoter followed by the eglA-mTNF-α fusion gene (see table 3).

TABLE 4
Sequences of the recA and eglA promoter at the 3′ region.

| | |
|---|---|
| recA promoter [SEQ ID NO 54] | 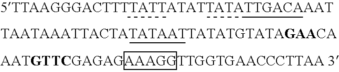 |
| eglA promoter [SEQ ID NO 55] | 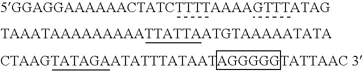 |

-10 and -35 promoter elements are underlined. The Shine-Dalgarno (SD) sequence is boxed. Sequences being mutated in accordance with the present invention are underlined with interrupted line. The Cheo box is presented in bold.

For mutation of the eglA and recA promoter, mutagenic primers containing an extra Cheo box flanked by 10-15 bases of the correct sequence were 5′ TATATTGACAAATGAACAAATGTTCATATAATTATATG 3′ [SEQ ID NO44] and 5′ CATATAATTATATGAACATTTGTTCATTTGTCAATATA 3′ [SEQ ID NO45] Primers to delete the Cheo box in the recA promoter region were 5′ TAATTATATGTATA_{deletion 12 bp}GAGAGAAAGGTTGG 3′ [SEQ ID NO 46] and 5′ CCAACCTTTCTCTC_{deletion 12 bp}TATACATATAATTA 3′ [SEQ ID NO 47] Primers to introduce a Cheo box in the eglA promoter region were 5′ TTTAAGGGACTTTGAACATATGTTCTTGACAAATTAAT 3′ [SEQ ID NO 48] and 5′ ATTAATTTGTCAAGAACATATGTTCAAAGTCCCTTAAA3′ [SEQ ID NO 49]. To verify the insertion or deletion of the Cheo box, the DNA fragments containing the introduced mutations were subcloned in pUC19 and the DNA sequence was determined with an automated laser fluorescent ALF Express sequencer (Amersham Pharmacia BioTech). Primers used for sequencing were the CY5-labeled M13 forward and reverse primers.

All general DNA manipulations in E.coli were carried out as described by Sambrook et al. (Sambrook, J. E. et al, 1989, Molecular cloning: a laboratory manual, 2^{nd} ed. Cold Spring Harbor Laboratory Press, Cold Spring Harbor, N.Y.). Restriction endonucleases and DNA-modifying enzymes were purchased from Roche Diagnostics (Brussels, Belgium), GIBCO BRL (Gaithersburg, Md.) and Eurogentec (Seraing, Belgium) and used as indicated by the suppliers. DNA plasmid isolations from E.coli were performed with the Wizard Plus SV miniprep kit (Promega Inc., Madison, Wis.).

E. coli was transformed using chemically competent cells obtained with the RbCI method. Transformation of C. acetobutylicum DSM792 was carried out by electroporation as published (Nakotte, S. et al, M., 1998, Appl. Microbiol. Biotechnol. 50:564-567).

### EXAMPLE 3

### Irradiation of Bacteria

Recombinant bacteria were grown until early log phase (OD₆₀₀ₙₘ=±0.3). At this time point cultures were divided into two sets, one of which was exposed to radiation while the other was mock irradiated and used as a control. Bacteria were exposed to 2 Gy with a ⁶⁰Cobalt unit at a dose-rate of 0.9 Gy/min. After irradiation, bacteria were incubated anaerobically at 37° C. and samples were taken at different time intervals after exposure. Each experiment was independently repeated three times.

### EXAMPLE 4

### Analysis of mTNF-α Secretion

The amount of mTNF-α secreted by recombinant clostridia was quantified using ELISA. Supernatant taken from irradiated and non-irradiated cultures was diluted 10-fold in phosphate buffered saline plus 7.5% bovine serum albumin and 100 μl aliquots were put in a 96-well microtiter plate in duplicate. Further manipulations were done according to the manufacturer's protocol (DiaMed EuroGen, Tessenderlo, Belgium).

Concentrations of secreted mTNF-α were calculated and compared for the irradiated and non-irradiated cultures. The level of radio-induced mTNF-α production was expressed as the relative increase in mTNF-α concentration of irradiated samples compared with the corresponding non-irradiated samples. Immunoblot analysis with polyclonal rabbit anti-mTNF-α antibodies was carried out by the method of Van Mellaert et al. (Van Mellaert, L. et al,. 1994, Gene 150:153-158.).

### EXAMPLE 5

### Reverse Transcriptase-PCR (RT-PCR)

To prove that induction of mTNF-α was the result of an increase in promoter activity, RT-PCR was performed on RNA isolated from irradiated and non-irradiated bacterial cultures. One hour after radiotherapy, aliquots of 4 ml culture were taken and RNA was extracted using the “RNeasy mini kit” from QIAGEN (Valencia, Calif.) as previously described (Nuyts, S., et al., 2001, J. Microbiol. Methods. 44:235-238.). RNA concentration was determined spectrophotometrically. To avoid DNA contamination which could result in mTNF-cDNA transcription from the plasmid, 1 μg RNA was digested with Fnu4HI which cleaves the mTNF-cDNA at position 82 and 213 and an additional DNase treatment was carried out. After heat-inactivation of the enzymes, 200 U Murine-Moloney Leukemia Virus-Reverse Transcriptase (M-MLV-RT) (GIBCO BRL, Gaithersburg, Md.) were added to the RNA together with 0.8 μl dNTPs (5 mM each), 4 μl 5×RT-buffer, 2 μl reverse primer (10 pmol/μl) and 2 μl Dithiothreitol (0.1 M) in a total volume of 20 μl. After 1 hour incubation at 37° C., the resulting cDNAs were amplified using PCR. 5 μl of the RT-mixture was added to 8.5 μl reverse primer, (10 pmol/μl), 7 μl forward primer (10 pmol/μl), 2.5 μl dNTPs (5 mM each), 0.5 U JumpStart Taq DNA Polymerase (Sigma, Sigma Chemical Co., St. Louis, Mo.) and 4.5 μl 10×buffer in a total volume of 50 μl. After 40 PCR-cycles (10 min 95° C., 30 sec 95° C., 2 min 40° C., 30 sec 72° C., 5 min 72° C.) 1 μl aliquots were run on 1% agarose gels. Primers used for amplification of mTNF-α were: Forward primer: 5′ GTAAGATCAAGTAGTCAA 3′ [SEQ ID NO 50] and Reverse primer: 5′ CAGAGCAATGACTCCAAA 3′ [SEQ ID NO 51]. To verify the absence of any DNA contamination all samples underwent the same RT-PCR reactions, without the addition of M-MLV-RT. To ensure equal amounts of RNA in all samples, an internal fragment of Clostridium acetobutylicum 16S rRNA was amplified using RT-PCR to function as internal standard. Primers used for amplification of 16S rRNA were

| | |
|---|---|
| Forward primer: | |
| 5′GGAGCAAACAGGATTAGATACC 3′ and | [SEQ ID NO 52] |
| Reverse primer: | |
| 5′ TGCCAACTCTATGGTGTGACG 3′. | [SEQ ID NO 53] |

### EXAMPLE 6

### Mutation of the recA and eglA Promoter

After introduction of mutations in the vectors pIMP-eglA-mTNF-α and pIMP-recA-mTNF-α by PCR mutagenesis, mutations were verified by sequence analysis and restriction digestion. Therefore, a 605 bp fragment of both plasmids containing the mutated sequence were subcloned in pUC19 digested with HindII.

Since both eglA and recA promoter are functional in E.coli, it was possible to test activity of the mutated promoters by determination of expression and secretion of mTNF-α. Lysates and supernatants were analysed via Western blot analyses using rabbit anti-mTNF-α polyclonal antibodies and alkaline phosphatase conjugated anti-rabbit antibodies (Sigma, Sigma Chemical Co., St. Louis, Mo.). In both cell lysates and supernatants clearly the presence of mTNF-α was demonstrated by all recombinant bacteria containing the different constructs, proving that the mutated promoters were still functional.

After introduction of the recombinant plasmids into Clostridium via electroporation, presence of mTNF-α was again demonstrated in supernatants and lysates via immunoblotting.

### EXAMPLE 7

### Results on the Analysis of mTNF-α Secretion

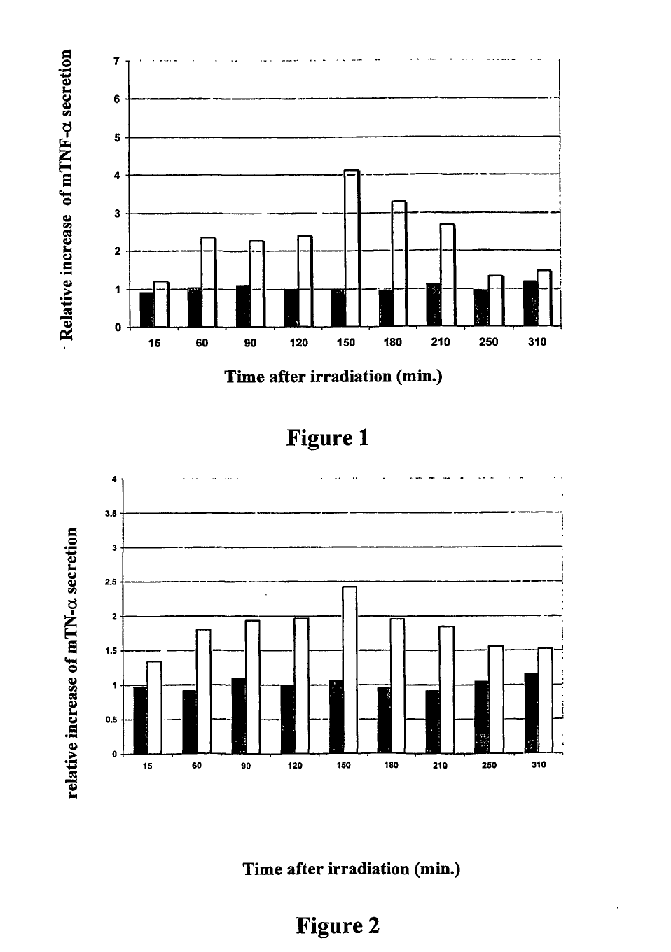

ELISA analysis was used to quantify mTNF-α secretion by recombinant clostridia. As shown earlier, the ‘wild-type’ recA promoter gives a 1.44-fold increase of mTNF-α secretion after a single dose of 2 Gy. After deletion of the Cheo box from the recA promoter region, no significant increase of mTNF-α secretion was measured after irradiation compared with the control samples (FIG. 1). However, incorporation of an extra Cheo box in the recA promoter region, resulted in a 4.12-fold increase in mTNF-α secretion 2.5 hrs after a single dose of 2 Gy (FIG. 1). 1.5 hrs after radiation, the increase in mTNF-α secretion increased by a factor of about 2.3.

Irradiation of the recombinant bacteria containing the pIMP-eglA-mTNF construct, resulted in no increase in mTNF-α secretion, confirming the constitutive properties of the eglA promoter (FIG. 2). However, when a Cheo box was incorporated in the eglA promoter region, a 2.42-fold increase was seen 2.5 hrs after 2 Gy irradiation (FIG. 2). Again, 1.5 hrs after radiation, the increase in mTNF-α secretion was 1.93.

The present example demonstrates that strong constitutive promoters such as the eglA promoter can be made radio-inducible by introducing a Cheo box in the promoter region. This implies that secretion of high doses therapeutic proteins like TNF-α can be controlled by ionising irradiation. Since the Cheo box is functional in the eglA promoter, independently of its natural sequence context, it will be possible to radio-induce other clostridial promoters, which might even be stronger. Moreover the addition of more Cheo boxes will increase repression and hence augment inducibility further.

### EXAMPLE 8

### Results on the RT-PCR

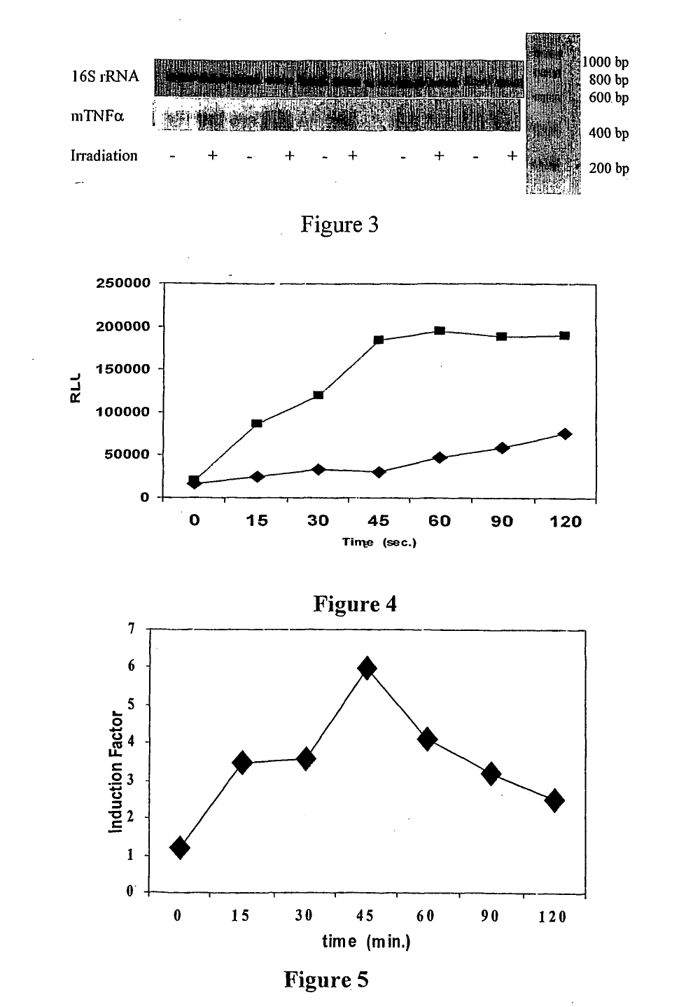

Reverse transcriptase-PCR was carried out to prove that the increase in mTNF-α secretion was the result of an increase in promoter activity. 1 μl of the PCR-mixture was put on gel (FIG. 3). The upper panel represents the 650 bp internal fragment of 16S rRNA, which was amplified to ensure equal amounts of RNA were used in each PCR reaction. The lower panel represents the 470 bp internal fragment of mTNF-α, which was amplified. As shown the upper panel in FIG. 3, equal amounts of RNA were used in all reactions. When mTNF-α was amplified, both for the constructs with the eglA promoter with a Cheo box introduced (lanes 1 and 2), the ‘wild-type’ recA promoter (lanes 3 and 4) and the recA promoter with the extra Cheo box (lanes 5 and 6), the samples from non-irradiated conditions result in a weaker band than the samples irradiated, indicating that more mRNA was present in the irradiated samples. For the constitutive eglA promoter (lanes 8 and 9) and the recA promoter with a deletion of the Cheo box (lanes 10 and 11), no difference can be seen between the irradiated and the non-irradiated samples. For the control samples, both the recA promoter with an extra Cheo box and the eglA promoter containing a Cheo box, showed a weaker band than the corresponding ‘wild-type’ promoters. This weaker signal is attributed to lower transcription levels because of higher repression levels under non-induced conditions. The reverse is seen for the recA promoter with a deletion of the Cheo box: a higher signal in the non-irradiated samples for the mutated promoter could be seen in comparison with the ‘wild-type’ promoter. This higher signal is the result of the absence of repression.

This experiment shows that the addition of repressor responsive elements result lower basal expression levels.

The absence of any band in the samples to which no reverse transcriptase was added, confirmed there was no DNA contamination present in none of the samples.

### EXAMPLE 9

### Radiation Induced Expression in Gram Negative Bacteria

Construction of the recombinant plasmid were carried out and then transformed in E. coli TG1 (supE hsdΔ5 thi Δ(lac-proAB) F′ [traD36 pro AB⁺ lacl^{q} lacZΔM15] strain. E.coli cells were routinely grown at 37° C. in LB medium under aerobic conditions by shaking. Chromosomal DNA was extracted from TG1 E. coli cells using a wizard genomic DNA purification kit (Promega). To isolate the recA promoter, PCR was carried out by using this chromosomal DNA with specific oligonucleotides designed based on the E coli DNA sequence with EMBL Accession number EC V00328, incorporating restriction sites (KpnI and Bg/II) at the 5′ end of each primers (ECRECA1 5′-TAGGTACCGTCTGGTTTGCTTGC-3′ [SEQ ID NO 56]; ECRECA2 5′-TAAGATCTCATGCCGGGTAATACC-3′ [SEQ ID NO 57]. DNA fragments of the expected size were amplified and cloned into pGEM-T Easy vector (Promega) resulting into plasmid pRecA-GEM-T Easy. This plasmid was digested with KpnI and Bg/II to adapt the termini for in-frame insertion of the recA promoter into Kpn1-Bg/II sites in the pSp-luc+NF fusion vector (Promega). The resultant expression plasmid was designated pRecA-Luc+NF (table 1). All restriction enzymes, T4 DNA ligase and polymerase were from GIBCO BRL (Gaithzersburg, Md.), and Eurogentec (Seraing,Belgium) and used as indicated by the supplier. The condition used for plasmid DNA extractions, restriction endonuclease digestion, agarose gel electrophoresis and isolation and ligation of DNA fragments have been carried out according to standard protocol (Sambrook, et al. 1989). Plasmid DNA was isolated from E. coli with a Wizard Plus SV miniprep kit (Promega Inc, Madison, Wis.). Plasmid pRecA-GEM-T Easy was transformed into chemically competent E. coli cells obtained with the RbCI method. Selection of the transformants carrying the appropriate plasmid was made on the bases of ampicillin resistance and Blue/white colony on the LB agar plate containing ampicillin and 5-bromo-4chloro-3-indolyl-B-D-galactopyranoside (X-gal) and IPTG. Ampicillin, the X-gal and IPTG were used at a final concentration of 50, 50 and 200 μg/ml respectively.

The sequence of cloned products was verified by automatic sequencing using the thermo sequanase fluorescent labeled Amersham cycle sequencing kit based on Sanger dideoxy-method for sequencing in the ALFexpress®Autoread® Sequencer (Amersham Pharmacia Biotech) and compared with the deposited sequence

Luciferase Reporter Assays: Cultures of transformed bacteria with pRecA-Luc+NF were grown to OD600 of about 0.3 (˜1×10.8.cells/ml). Subsequently, these cultures were divided into two fractions. One fraction was irradiated with UV at a dose of 254 nm for 60 sec in a Petri-dish. The irradiated culture was reintroduced into the tube) and similarly as the non-irradiated culture continued to grow at 37° C. with shaking. The aliquot of the cultures were collected every 15 minutes till 1 hour followed with every 30 minute till 120 min., and the luciferase activity was determined.

20 μl aliquot of sample and 100 μl of luciferase assay reagent were place in black 96 well plate and placed in to luminometer chamber at a temperature control of 20° C. enzyme activity were measured using a Packard Lumicount micro plate luminometer. All measurements were taken at 0.5 and 2 sec per well-read length. Luminescence values are presented as relative light units (RLU)(as per the particular instrument's output). The induction factor was calculated by dividing the enzyme activity of an induced sample displayed at the times indicated by that of a matched non-induced sample. The induction factor of 1.0 represents no induction. Each induction experiment was repeated three or more times and the mean values are shown in table 5 and FIGS. 4 and 5.

TABLE 5
activity of a reporter gene under the control of a radiation
inducible promoter.

| | Non | | | |
|---|---|---|---|---|
| | Exposed | Exposed | non exposed | exposed |
| | ((relative | (relative light | (induction | (induction |
| Time (sec) | light units | units) | factor) | factor) |
| 0 | 16200 | 19700 | 1 | 1.21 |
| 15 | 24900 | 86000 | 1 | 3.46 |
| 30 | 33700 | 119800 | 1 | 3.55 |
| 45 | 30800 | 184400 | 1 | 5.99 |
| 60 | 47800 | 195200 | 1 | 4.08 |
| 90 | 58900 | 188400 | 1 | 3.20 |
| 120 | 75700 | 190400 | 1 | 2.52 |

These results show that the radiation induced expression of a heterologous gene under the control of the RecA. promoter leads to a five fold higher relative induction than the basal expression under non irradiated conditions.

The present example shows that the RecA promoter alone is sufficient to preform radiation induced expression outside its natural genomic DNA environment. Since the radiation induced expression in both Gram negative bacteria and Gram positive bacteria is determined by the repressor binding elements.

1. An isolated and purified polynucleotide comprising at least one first sequence element inserted in a second sequence element wherein the first sequence element is a repressor binding element of a promoter which is inducible by DNA damaging agents or conditions and wherein the second sequence element is a promoter sequence.

2-41. (cancelled).

42. The polynucleotide of claim 1, wherein the promoter sequence of the second sequence element is from a promoter which is not inducible by a DNA damaging agent or condition.

43. The polynucleotide of claim 1, wherein the promoter sequence of the second sequence element is from a promoter which is inducible by a DNA damaging agent or condition.

44. The polynucleotide of claim 1, wherein said polynucleotide is positioned 5′ to a nucleotide sequence suitable for the introduction of a third sequence element.

45. The polynucleotide of claim 1, wherein the insertion of a first sequence element occurs between about 46 base pairs and about 106 base pairs upstream of the ribosome binding site of the second sequence element.

46. The polynucleotide of claim 42, wherein the non-inducible promoter is a constitutive promoter or an inducible promoter.

47. The polynucleotide of claim 42, wherein the non-inducible promoter is a bacterial promoter.

48. The polynucleotide of claim 47, wherein the bacterial promoter is from gram positive bacteria.

49. The polynucleotide of claim 47, wherein the bacterial promoter is from gram negative bacteria.

50. The polynucleotide of claim 47, wherein the bacterial promoter is an EglA promoter of Clostridium sp.

51. The polynucleotide of claim 43, wherein the inducible promoter is a bacterial promoter.

52. The polynucleotide of claim 51, wherein the bacterial promoter is from gram positive bacteria.

53. The polynucleotide of claim 51, wherein the bacterial promoter is from gram negative bacteria.

54. The polynucleotide of claim 53, wherein the bacterial promoter is RecA.

55. The polynucleotide of claim 1, wherein the repressor binding element comprises a Cheo box consensus sequence as depicted in SEQ ID NO: 1.

56. The polynucleotide of claim 1, wherein the repressor binding element comprises a DinR box consensus sequence as depicted in SEQ ID NO: 2.

57. The polynucleotide of claim 1, wherein the repressor binding element comprises a sequence selected from the group of sequences depicted in SEQ ID 4 to SEQ ID 25.

58. The polynucleotide of claim 1, wherein the repressor binding element comprises a SOS box consensus sequence as depicted in SEQ ID NO3.

59. The polynucleotide of claim 1, wherein the repressor binding element comprises a sequence selected from the group of sequences depicted in SEQ ID 26 to SEQ ID 43.

60. The polynucleotide of claim 44, wherein the third sequence element encodes a protein with pharmaceutical properties or a protein which is able to convert an inactive compound into a pharmaceutically active compound.

61. The polynucleotide according to claim 60 wherein the protein with therapeutic properties is TNF-alpha (Tumour Necrosis Factor alpha).

62. A method of converting a promoter which is not inducible by DNA damaging agents or conditions into a promoter which is inducible by radiation, genotoxic compounds or DNA damaging compounds comprising the step of inserting at least one repressor binding element of a promoter which is inducible by a DNA damaging compound or condition into said non inducible promoter.

63. A method of increasing the induction level of a first promoter which is inducible by genotoxic compounds or conditions comprising the step of inserting at least one repressor binding element of said first promoter or of a second promoter which is inducible by a DNA damaging compound or condition into said first inducible promoter.

64. A method of decreasing the basal expression level of a first promoter which is inducible by genotoxic compounds or conditions comprising the step of inserting at least one repressor binding element of said first promoter or of a second promoter which is inducible by a DNA damaging compound or condition into said first inducible promoter.

65. A vector comprising a nucleotide sequence according to claim 1.

66. A bacterial host cell transfected with the vector of claim 65.

67. A bacterial host cell according to claim 66, wherein said cell is a facultative or obligate anaerobic bacterium.

68. A pharmaceutical composition comprising a cell according to claim 66 in admixture with at least one pharmaceutically acceptable carrier.

69. A pharmaceutical composition comprising a cell according to claim 67 in admixture with at least one pharmaceutically acceptable carrier.

70. A method for the in vitro production of recombinant proteins comprising the step of contacting a culture of host cells according to claim 66 with a DNA damaging compound or condition.
